(19) United States
(12) Patent Application Publication
(10) Pub. No.: US 2025/0090751 A1
(43) Pub. Date: Mar. 20, 2025

(54) ELECTRONIC DEVICE FOR COLLECTING DRUG INJECTION INFORMATION ACCORDING TO USE OF PATIENT-CONTROLLED ANALGESIA (PCA) DEVICE, CONTROL METHOD THEREFOR, AND SYSTEM

(71) Applicant: SEOUL NATIONAL UNIVERSITY R&DB FOUNDATION, Seoul (KR)

(72) Inventors: Cheol Woo JEONG, Seoul (KR); Hyung Chul LEE, Seoul (KR)

(73) Assignee: SEOUL NATIONAL UNIVERSITY R&DB FOUNDATION, Seoul (KR)

(21) Appl. No.: 18/294,414

(22) PCT Filed: Nov. 4, 2021
(86) PCT No.: PCT/KR2021/015925

§ 371 Date: Feb. 1, 2024

(30) Foreign Application Priority Data
Oct. 5, 2021 (KR) 10-2021-0131757

(51) Int. Cl. A61M 5/172; A61M 5/14

(52) U.S. Cl. CPC A61M 5/172; A61M 2005/1405; A61M 2205/3569; A61M 2205/502

(57) ABSTRACT

According to one embodiment of the present invention, an electronic device for collecting drug injection information according to the use of a patient-controlled analgesia (PCA) device comprises: a communicator; an input device including a connection terminal connected to the PCA device; and a controller for receiving a drug injection request signal through the input device, identifying drug setting information and drug injection information corresponding to the drug injection request signal based on receiving the drug injection request signal, and transmitting the drug setting information and the drug injection information through the communicator.

### FIELD OF THE DISCLOSURE

The present invention relates to an electronic device associated with a PCA device, and a control method and system thereof.

### DESCRIPTION OF RELATED ART

Patients undergoing surgery complain of varying degrees of pain after surgery. In this case, generally, according to the patient's demand for pain relief, the medical staff prescribes a basic dose of a drug such as an analgesic to control the pain, and determines the type and dosage of an additional drug according to the result thereof. This method has a problem in that the satisfaction of patients and medical staff is very low, because the time interval from the onset of pain to obtaining appropriate pain relief is too long.

In order to solve this problem, a PCA device was developed that allows the patient to decide the timing and dosage of the drug by himself, and afterwards, the spread of the PCA device improved patient satisfaction in postoperative pain treatment and significantly reduced the number of medical personnel involved.

The PCA device provides continuous intravenous infusion at the basal infusion rate by using a pump that mechanically or electronically controls the drug stored in the device, and the patient operates the PCA device to additionally inject a bolus dose according to the patient's needs.

The drug injection information, including the amount of injected drug, the number of times and intervals of pressing the self-regulation button of the PCA device and the like, serves as a basis for adjusting the drug dose of the PCA device in the same surgical patient or patients with similar conditions in the future, and become the main data in various research and developments related to the PCA device.

However, mechanical PCA devices do not have a data communication function, and some electronic PCA devices with communication functions are not only expensive because they are equipped with large-capacity batteries due to power management problems, but also cause the PCA device itself to be excessively large and heavy, and therefore, there is a problem in that the patient's activities after surgery have been limited. In this way, the acquisition of a patient's drug injection information is limited due to the absence or limitation of data storage or communication functions in the PCA device, and thus, the situation is that it is difficult to utilize the PCA device, such as monitoring and controlling the patient's pain and the corresponding drug use level.

### SUMMARY

An object of the present invention is to provide an electronic device, a control method thereof and a system for improving the function of a PCA device.

Another object of the present invention is to provide an Internet of Things (IoT) button device that enables the storage and real-time use of drug injection history data by transmitting drug usage information of a patient to a network.

The electronic device for collecting drug injection information according to the use of a patient-controlled analgesia (PCA) device according to an exemplary embodiment of the present invention includes a communicator; an input device including a connection terminal connected to the PCA device; and a controller for receiving a drug injection request signal through the input device, identifying drug setting information and drug injection information corresponding to the drug injection request signal based on receiving the drug injection request signal, and transmitting the drug setting information and the drug injection information through the communicator.

The input device may further include a switch which is connected to the connection terminal, and the controller may receive the drug injection request signal based on receiving a user input of pressing the switch.

The input device may further include a cable which is connectable to the connection terminal and an external switch which is connected to the cable and detachable from the PCA device, and the controller may receive the drug injection request signal based on receiving a user input of pressing the external switch.

The PCA device may include a first PCA device and a second PCA device, wherein the connection terminal may include a first connection terminal which is connectable with the first PCA device and a second connection terminal which is connectable with the second PCA device, wherein the input device may further include a switch which is connected to the first connection terminal, and wherein the switch may be deactivated based on the connection of the second PCA device to the second connection terminal.

The controller may convert an operation mode of the electronic device from a first mode to a second mode with higher power consumption than the first mode based on receiving a drug injection request signal through the input device, and convert from the second mode to the first mode, after transmitting the drug setting information and drug injection information through the communicator according to the drug injection request signal.

The electronic device according to an exemplary embodiment of the present invention may further include a display, wherein the controller displays a GUI (Graphic User Interface) indicating the drug setting information on the display, and receives a user input for setting the drug setting information by using the GUI.

The electronic device according to an exemplary embodiment of the present invention may further include a storage, wherein the controller saves the identified drug setting information and drug injection information in the storage.

The controller may transmit a drug injection signal to the PCA device based on the identified drug setting information.

The method for controlling an electronic device for a patient-controlled analgesia (PCA) device according to an exemplary embodiment of the present invention may include the steps of receiving a drug injection request signal; identifying drug setting information and drug injection information corresponding to the drug injection request signal based on receiving the drug injection request signal; and transmitting the drug setting information and drug injection information.

The system for collecting drug injection information according to the use of a patient-controlled analgesia (PCA) device according to an exemplary embodiment of the present invention may include the PCA device; an electronic device which is connected to the PCA device; and a server which is connected to communicate with the electronic device, wherein the electronic device includes a communicator; an input device including a connection terminal connected to the PCA device; and a controller for receiving a drug injection request signal through the input device, identifying drug setting information and drug injection information corresponding to the drug injection request signal based on receiving the drug injection request signal, and transmitting the drug setting information and the drug injection information through the communicator to the server, and wherein the server receives the drug setting information and drug injection information from the electronic device through a server communicator.

According to an exemplary embodiment of the present invention, by collecting the patient's current pain and pain relief level in real time, the medical staff can remotely grasp the patient's condition and needs to improve the efficiency and quality of treatment.

According to an exemplary embodiment of the present invention, by using a large amount of information collected from the PCA device, it is possible to contribute to the improvement of medical treatment or research and development, such as developing an algorithm that proposes an appropriate set value and use method of a drug.

According to an exemplary embodiment of the present invention, the operation of the electronic device and the PCA device of the present invention are physically separated such that the safety of the PCA device can be increased while collecting useful information for generating a drug injection algorithm.

### BRIEF DESCRIPTION OF THE DRAWINGS

FIG. 1 is a diagram illustrating an electronic device according to an exemplary embodiment of the present invention.

FIG. 2 is a block diagram illustrating the configuration of an electronic device according to an exemplary embodiment of the present invention.

FIG. 3 is a diagram illustrating the operation flowchart of an electronic device according to an exemplary embodiment of the present invention.

FIG. 4 is a diagram illustrating the operation of an electronic device according to an exemplary embodiment of the present invention.

FIG. 5 is a diagram illustrating the operation of an electronic device according to another exemplary embodiment of the present invention.

FIG. 6 is a diagram illustrating a flowchart of the overall operation of a system according to an exemplary embodiment of the present invention.

### DETAILED DESCRIPTION OF THE INVENTION

Hereinafter, preferred exemplary embodiments according to the present invention will be described in detail with reference to the accompanying drawings. The detailed description set forth below in conjunction with the accompanying drawings is intended to describe the exemplary embodiments of the present invention and is not intended to represent the only exemplary embodiments in which the present invention may be practiced. In order to clearly describe the present invention in the drawings, parts that are irrelevant to the description may be omitted, and the same reference numerals may be used for the same or similar components throughout the specification.

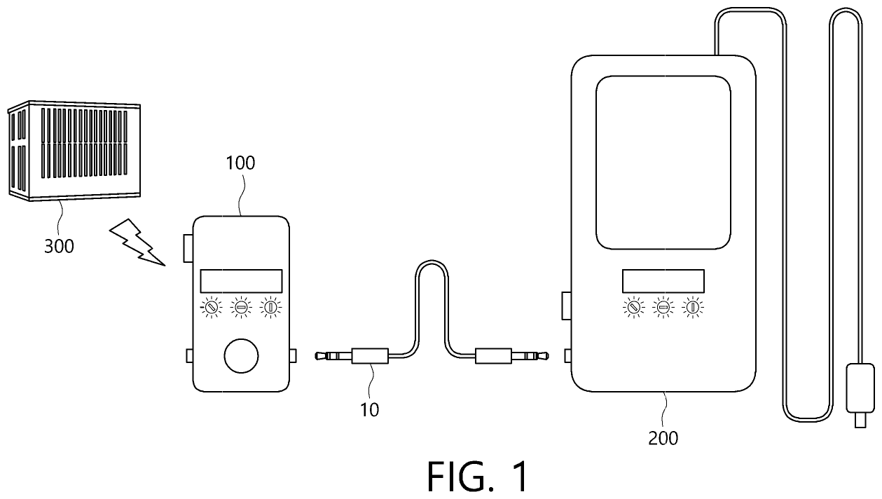

FIG. 1 is a diagram illustrating an electronic device according to an exemplary embodiment of the present invention.

FIG. 1 illustrates an electronic device 100 according to an exemplary embodiment of the present invention, an external device 200 which is connected to the electronic device 100 through a cable 10, and a server 300 which is communicatively connected to the electronic device 100. The external device 200 according to an exemplary embodiment of the present invention means a PCA device.

As described above, the electronic device 100 according to an exemplary embodiment of the present invention is a device which has been devised to solve the communication function problems of the conventional PCA device. Even if the PCA device itself does not have a communication function, by connecting the PCA device and the electronic device 100 equipped with a communication function by using the cable 10, it is possible to transmit useful information that can be obtained by using the PCA device, such as the user's drug injection information, to the outside, such as the server 300.

That is, by connecting the electronic device 100 equipped with a communication function with the PCA device, the medical staff may monitor the user's drug injection situation in real time or utilize information obtained in the same or similar situations in the future by using the information transmitted from the electronic device 100.

According to an exemplary embodiment of the present invention, the medical staff may contribute to improvement of medical treatment or research and development by using information obtained from the PCA device.

Accordingly, the present invention provides an electronic device, a control method and a system thereof that increase the utilization of the PCA device. Hereinafter, the configurations of an electronic device according to an exemplary embodiment of the present invention will be described with reference to the drawings.

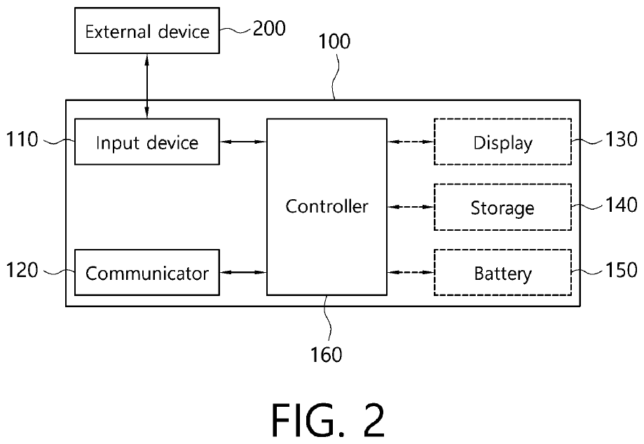

FIG. 2 is a block diagram illustrating the configuration of an electronic device according to an exemplary embodiment of the present invention.

An input device 110 includes at least one input means for generating input data in response to a user input of the electronic device 100 or a user input received from the external device 200, for example, the PCA device. The input device 110 may include an input terminal for connection with the external device 200, a button, a rotary switch, a keypad, a dome switch, a touch panel, a jog shuttle, a touch key and the like. More specifically, the input terminal may include a connector through which the electronic device 100 may be physically connected to the external device 200, such as a USB port, an ear jack terminal, an HDMI connector, an SD card connector or the like.

A communicator 120 performs communication with an external server (not illustrated). To this end, the communicator 120 may perform wireless communication such as 5th Generation communication (5G), Long Term Evolution-Advanced (LTE-A), Long Term Evolution (LTE) and Wireless Fidelity (Wi-Fi). The communicator 120 may use wireless communication such as radio frequency (RF), Zigbee, Bluetooth, Wi-Fi, Ultra WideBand (UWB) and Near Field Communication (NFC) as a communication method. The communicator 120 may be implemented as a wireless communication module that performs wireless communication with an AP, such as Wi-Fi, or a wireless communication module that performs one-to-one direct wireless communication, such as Bluetooth.

A display 130 displays display data associated with the operation of the electronic device 100. The display 130 may include a Liquid Crystal Display (LCD), a Light Emitting Diode (LED) display, an Organic LED (OLED) display, a Micro Electro Mechanical Systems (MEMS) display and an electronic paper display. The display 130 may be combined with the input device 110 and implemented as a touch screen.

A storage 140 stores operating programs of the electronic device 100. The storage 140 includes a non-volatile storage which is capable of preserving data regardless of whether power is provided, and a volatile memory in which data to be processed by a controller 160 is loaded and data cannot be preserved unless power is provided. Examples of the storage include flash-memory, hard-disc drive (HDD), solid-state drive (SSD), read-only memory (ROM) and the like, and examples of the memory include buffer, random access memory (RAM) and the like.

A battery 150 may supply power to at least one component of the electronic device 100.

A controller 160 may execute software such as a program to control at least one other component (e.g., hardware or software component) of the electronic device 100 connected to the controller 160, and perform various data processing or operations.

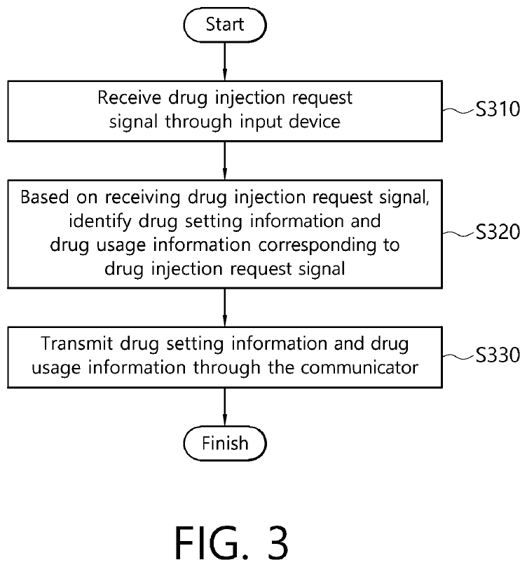

FIG. 3 is a diagram illustrating the operation flowchart of an electronic device according to an exemplary embodiment of the present invention.

According to an exemplary embodiment of the present invention, the controller 160 may receive a drug injection request signal through the input device 110 (S310).

The input device 110 may include a switch which is connected to the connection terminal, for example, a switch which is built into the electronic device 100 or an external switch which is detachable from the PCA device, which is the external device 200, and connected to the connection terminal. According to an exemplary embodiment, the external switch may be connected to a cable, and the external switch may be operated by inserting the cable into a connection terminal.

The drug injection request signal is a signal requesting drug injection, and when a user requests drug injection, such as pressing a switch, the controller 160 may receive the drug injection request signal through the input device 110.

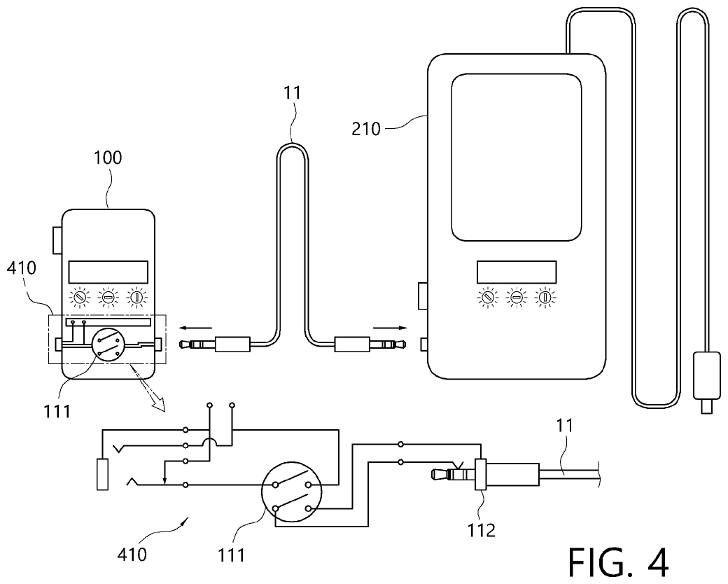

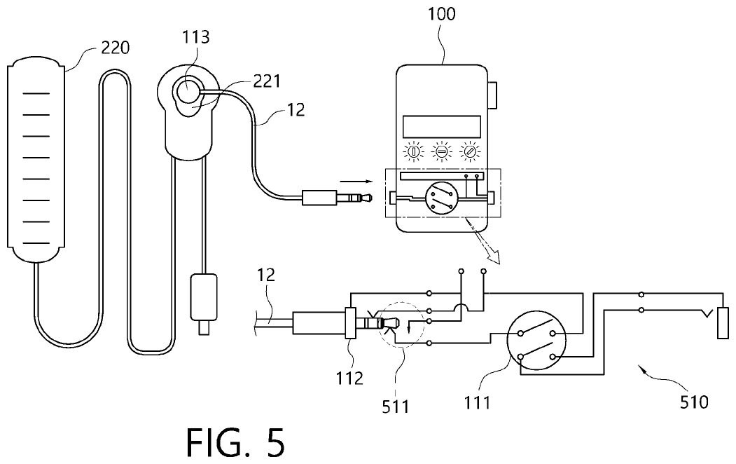

According to an exemplary embodiment, when the electronic device 100 is connected to the electronic PCA device, the electronic device 100 may receive a drug injection request signal from a user input of pressing a switch built in the electronic device 100. When the electronic device 100 is connected to the mechanical PCA device, a drug injection request signal may be received from a user input of pressing an external switch attached to the mechanical PCA device. Different input methods according to the types of PCA devices are described in detail in FIGS. 4 and 5.

According to an exemplary embodiment of the present invention, the controller 160 may identify drug setting information and drug injection information corresponding to the drug injection request signal based on receiving the drug injection request signal (S320).

Since the user may have pressed the switch with the intention of using the PCA device, this is a task to database the usage information of the PCA device.

More specifically, the drug setting information includes a bolus dose of the drug, a basal infusion rate of the drug, a lock-out time and the like. The lock-out time is set to prevent excessive use of the PCA device, and it refers to a time limiting additional injection of the drug even if there is a user input for requesting injection of the drug again within a set time.

The controller 160 may receive drug setting information that is set in the PCA device through the input device 110 such as a rotary switch or a touch panel of a display, or through a cable connected to the PCA device. Alternatively, there is no limitation on the identification method of the drug setting information, such as connecting and receiving the memory containing the drug setting information of the PCA device to the electronic device 100. For example, the electronic device 100 may further include a display 130, and the controller 160 may display a GUI (Graphic User Interface) indicating the drug setting information on the display and receive a user input for setting the medication setting information by using the GUI.

The drug injection information includes the amount of drug injected, the interval between the previous injection time and the current injection time of the drug, the validity of the user input identified from the lock-out time or the injection time interval, the time the user input is received, the number of times the user input is received and the like.

According to an exemplary embodiment of the present invention, the controller 160 may transmit drug setting information and drug injection information through a communicator (S330).

The controller 160 may transmit the drug setting information and drug injection information identified in S320 to an external device such as a server. The information transmitted by the controller 160 is stored in the server and becomes a basis for adjusting the drug setting information of the PCA device in the same or similar case in the future, and becomes the major data for improving the drug injection algorithm. However, there may be cases where the drug injection information is not valid, such as when the user unnecessarily repeatedly presses a switch. In this case, the controller 160 may attach an “invalid event” tag to the user input that is repeatedly received within the lock-out time period and transmit information such as an invalid event occurrence time.

According to an exemplary embodiment of the present invention, by using a large amount of information collected from the PCA device, it is possible to contribute to the improvement of medical treatment or research and development, such as developing an algorithm that proposes an appropriate set value and use method of a drug.

FIG. 4 is a diagram illustrating the operation of an electronic device according to an exemplary embodiment of the present invention.

FIG. 4 illustrates a first PCA device 210, which is one of the external devices 200, an electronic device 100 connected to and operating with the first PCA device 210, and a partial circuit diagram 410 inside the electronic device 100. The first PCA device 210 according to the present exemplary embodiment refers to a PCA device that operates electronically.

According to an exemplary embodiment of the present invention, the electronic device 100 may further include a switch 111. Looking at the circuit diagram 410, the switch 111 may be configured as a double-pole single-throw (DPST) switch. The DPST switch is designed such that two single-pole single-throw (SPST) switches operate simultaneously with one switch.

According to an exemplary embodiment of the present invention, the electronic device 100 and the first PCA device 210 may be connected by using the cable 11. In addition, upon receiving a user input of pressing the switch 111, one SPST switch is connected to the controller 160 and transmits a drug injection request signal. That is, the controller 160 may receive a drug injection request signal based on a user input of pressing the switch 111. As described above in S320 and S330 of FIG. 3, the controller 160 may perform an operation such as identifying related information based on receiving a drug injection request signal and transmitting the same to a server. Another SPST switch transmits a drug injection request signal to the first PCA device 210 through the cable 11 connected to the connection terminal 112. Upon receiving the drug injection request signal, the first PCA device 210 injects the drug into the user according to the set drug setting information.

According to an exemplary embodiment of the present invention, since a switch for operating the first PCA device 210 and a switch for transmitting usage information of the first PCA device 210 are implemented separately, it is possible to secure the stability of using the PCA device.

However, the present invention is not limited to the above configuration, and when the switch 111 is mounted on the cable 11 and receives a user input of pressing the switch 111, a signal is transmitted to the electronic device 100 and the first PCA device 210, respectively, or the switch 111 is built into the first PCA device 210 such that the electronic device 100 may be implemented in various ways, such as receiving a drug injection request signal from the first PCT device 210.

Additionally, according to another exemplary embodiment of the present invention, upon receiving a drug injection request signal, the controller 160 may transmit a drug injection signal to the first PCA device 210 based on the identified drug setting information.

According to an exemplary embodiment of the present invention, even if the first PCA device 210 is not equipped with a function that is capable of communicating with the outside, such as a communication module, it is possible to obtain useful information including whether the user is using the first PCA device 210 by using the electronic device 100.

FIG. 5 is a diagram illustrating the operation of an electronic device according to another exemplary embodiment of the present invention.

FIG. 5 illustrates is a second PCA device 220, which is one of the external devices 200, an electronic device 100 connected to and operating with the second PCA device 220, and a partial circuit diagram 510 inside the electronic device 100. The second PCA device 220 according to the present exemplary embodiment refers to a PCA device that operates mechanically.

In the case of the second PCA device 220, drug setting information is set in advance, and the user uses the same by pressing a drug injection button 221 of the second PCA device 220 as necessary. In order to receive a drug injection request signal from the second PCA device 220 without a communication function, it is possible to use an external switch 113 which is detachable from the drug injection button 221 of the second PCA device 220 according to an exemplary embodiment of the present invention. The external switch 113 is connected to an end of the cable 12, and the cable 12 may be connected to a connection terminal 112 of the electronic device 100.

The electronic device 100 may receive a user input of pressing the external switch 113 from the cable 12 which is connected to the external switch 113. Looking at the circuit diagram 510, the switch 113 may be configured as a single-pole single-throw (SPST) switch.

According to an exemplary embodiment of the present invention, the controller 160 may receive a drug injection request signal when receiving a user input of pressing the external switch 113. As described above in S320 and S330 of FIG. 3, the controller 160 may perform an operation such as transmitting related information to a server on the basis of the reception of a drug injection request signal.

As described above with reference to FIG. 4, when the electronic device 100 includes the built-in switch 111 for the first PCA device 210, there may be cases in which the built-in switch 111 may be accidentally pressed while using the electronic device 100 in connection with the second PCA device 220. In this case, wince the drug injection request signal received from the built-in switch 111 is an invalid event, it is necessary to deactivate the built-in switch 111. Therefore, in an exemplary embodiment of the present invention, a method for solving this problem is described. Looking closely at the circuit diagram 510, the cable 12 consists of a three-pole jack. When the cable 12 is inserted into the connection terminal 112 connected to the second PCA device 220, the circuit connecting the control unit 160 and the built-in switch 111 is cut off by opening the contact points for connecting the controller 160 and the built-in switch 111 as shown in an end terminal area 511 of the 3-pole jack. Therefore, when connected to the second PCA device 220, it is possible to prevent a drug injection request signal from being input even if the built-in switch 111 is accidentally pressed.

However, the present invention is not limited to the above configuration, and the controller 160 may be implemented in various ways, such as identifying that the electronic device 100 and the second PCA device 220 are connected, and controlling an internal switch 111 to be deactivated based thereon.

According to an exemplary embodiment of the present invention, it is possible to obtain useful information including whether the second PCA device 220 that is not equipped with a communication function is used by using the electronic device 100.

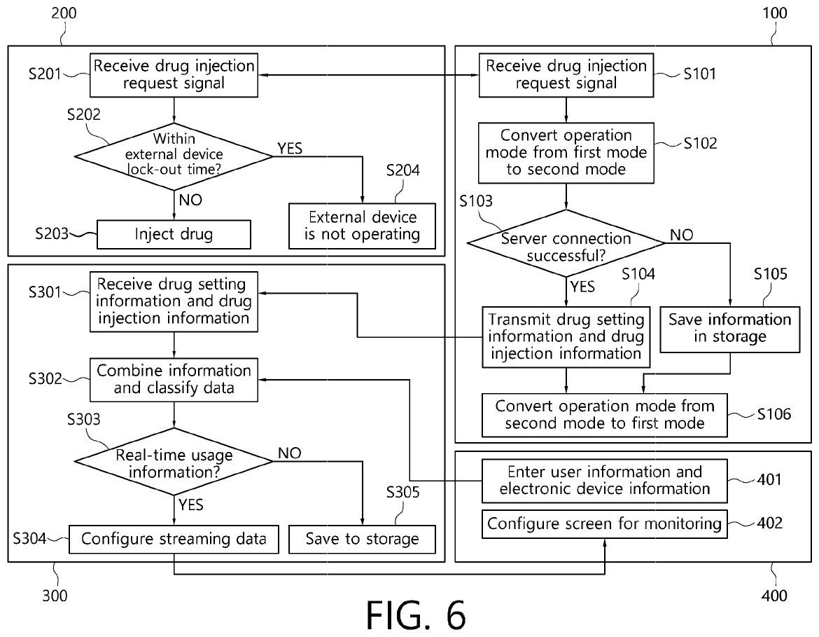

FIG. 6 is a diagram illustrating a flowchart of the overall operation of a system according to an exemplary embodiment of the present invention.

This drawing shows a flowchart of the organic operation between an electronic device 100, an external device 200, a server 300 and a user program 400. Hereinafter, it will be reviewed in order.

As described in S310 of FIG. 3, the electronic device 100 receives a drug injection request signal through the input device 110 (S101).

The controller 160 may convert an operation mode of the electronic device 100 from a first mode to a second mode with higher power consumption than the first mode on the basis of the reception of a drug injection request signal through the input unit 110 (S102). In this case, the first mode may be a sleep mode or power saving mode that reduces power consumption below a predefined value. When receiving a drug injection request signal or turning on the electronic device 100, the controller 160 may change the operation mode from the first mode to the second mode. By using this function, the electronic device 100 may save the battery 150.

The controller 160 may perform the operation of S320 of FIG. 3 in the second mode, which is a normal power consumption mode, and access the server to transmit the same to the server 300. If the connection to the server is successful (Yes in S103), the controller 160 may transmit drug setting information and drug injection information to the server 300 (S104).

If the connection to the server is not successful (No in S103), the information may be stored in the storage 140 in case the information is transmitted after succeeding in the server connection in the future (S105). In this case, the information stored in the storage 140 may include not only drug setting information and drug injection information, but also the time when the electronic device 100 is turned on and starts operating.

After performing an operation according to whether the connection to the server is successful, the controller 160 may convert the operation mode from the second mode to the first mode again to save the battery 150 (S106).

As described above, the external device 200 includes a first PCA device 210 and a second PCA device 220. The external device 200 receives a drug injection request signal from the electronic device 100 (S201). If the received drug injection request signal is received after the lock-out time of the external device (No in S202), that is, if additional drug injection is required after a predefined time, the external device 200 may inject the drug (S203).

If the received drug injection request signal is received within the lock-out time of the external device (Yes in S202), the external device may not operate (S204). In this case, as described in S310 of FIG. 3, the controller 160 may identify that the received drug injection request signal is an invalid event and transmit information thereon.

The server 300 may receive drug setting information and drug injection information from the electronic device 100 (S301). The server may perform the combination of information received from the electronic device 100, user information received from the user program 400 and information of the electronic device 100 and/or classification of the information (S302). The operation of combining information or classifying information is not limited thereto, and it may be an operation for identifying and improving a drug treatment mechanism for a specific user. Alternatively, it may be an operation for generating information that is useful for research and development associated with drug treatment by combining and/or classifying information of a specific user, as well as an unspecified number of users who have undergone surgery similar to the user or have similar symptoms.

The server 300 may identify whether the received information is real-time usage information (S303), and if it is real-time usage information (Yes in S303), it may configure streaming data (S304). If the received information is not real-time usage information due to a server connection failure of the electronic device 100 (S303 No), the controller of the server 300 may store the information in the storage of the server (S305).

In addition, the user program 400 includes a management program 401 that inputs user information and information of the electronic device 100, and a monitoring program 402 that is used to download and visualize streaming data required for real-time monitoring from the server (300).

According to an exemplary embodiment of the present invention, through the development of the electronic device 100, the simplification of the external device 200 may not only provide convenience for the user to use the external device 200, but also contribute to improving medical treatment or research and development, such as developing an algorithm that suggests the appropriate configuration values and usage methods of a drug by using a large amount of information collected from the external device 200.

1. An electronic device for collecting drug injection information according to the use of a patient-controlled analgesia (PCA) device, comprising:
a communicator;
an input device comprising a connection terminal connected to the PCA device; and
a controller for receiving a drug injection request signal through the input device, identifying drug setting information and drug injection information corresponding to the drug injection request signal based on receiving the drug injection request signal, and transmitting the drug setting information and the drug injection information through the communicator.

2. The electronic device of claim 1, wherein the input device further comprises a switch which is connected to the connection terminal, and
wherein the controller receives the drug injection request signal based on receiving of a user input of pressing the switch.

3. The electronic device of claim 1, wherein the input device further comprises a cable which is connectable to the connection terminal and an external switch which is connected to the cable and detachable from the PCA device, and
wherein the controller receives the drug injection request signal based on receiving of a user input of pressing the external switch.

4. The electronic device of claim 1, wherein the PCA device comprises a first PCA device and a second PCA device,
wherein the connection terminal comprises a first connection terminal which is connectable with the first PCA device and a second connection terminal which is connectable with the second PCA device,
wherein the input device further comprises a switch which is connected to the first connection terminal, and
wherein the switch is deactivated based on the connection of the second PCA device to the second connection terminal.

5. The electronic device of claim 1, wherein the controller converts an operation mode of the electronic device from a first mode to a second mode with higher power consumption than the first mode based on receiving the drug injection request signal through the input device, and converts from the second mode to the first mode, after transmitting the drug setting information and drug injection information through the communicator according to the drug injection request signal.

6. The electronic device of claim 1, further comprising:
a display,
wherein the controller displays a GUI (Graphic User Interface) indicating the drug setting information on the display, and receives a user input for setting the drug setting information by using the GUI.

7. The electronic device of claim 1, further comprising:
a storage,
wherein the controller saves the identified drug setting information and drug injection information in the storage.

8. The electronic device of claim 1, wherein the controller transmits a drug injection signal to the PCA device based on the identified drug setting information.

9. A method for controlling an electronic device for a patient-controlled analgesia (PCA) device, comprising the steps of:
receiving a drug injection request signal;
identifying drug setting information and drug injection information corresponding to the drug injection request signal based on receiving the drug injection request signal; and
transmitting the drug setting information and drug injection information.

10. A system for collecting drug injection information according to the use of a patient-controlled analgesia (PCA) device, comprising:
the PCA device;
an electronic device which is connected to the PCA device; and
a server which is connected to communicate with the electronic device,
wherein the electronic device comprises a communicator; an input device comprising a connection terminal connected to the PCA device; and a controller for receiving a drug injection request signal through the input device, identifying drug setting information and drug injection information corresponding to the drug injection request signal based on receiving the drug injection request signal, and transmitting the drug setting information and the drug injection information through the communicator to the server, and
wherein the server receives the drug setting information and drug injection information from the electronic device through a server communicator.
